(19) United States
(12) Patent Application Publication
(10) Pub. No.: US 2004/0019027 A1
(43) Pub. Date: Jan. 29, 2004

(54) Method of treating cerebrotendinous xanthomatosis

(76) Inventors: Barry Forman, Irvine, CA (US); Isabelle Dussault, Thousand Oaks, CA (US)

Correspondence Address: PERKINS COIE LLP, POST OFFICE BOX 1208, SEATTLE, WA, 98111-1208, US

(21) Appl. No.: 10/412,659

(22) Filed: Apr. 11, 2003

Related U.S. Application Data
(60) Provisional application No. 60/371,701, filed on Apr. 12, 2002.

(51) Int. Cl. A61K 31/573

(52) U.S. Cl. 514/179

(57) ABSTRACT

The present invention provides methods for preventing or treating disorders associated with the degradation of cholesterol and bile alcohols through the use of ligands that interact with pregnane X receptors (PXR). In a preferred embodiment, PXR agonists are used to treat disorders associated with sterol 27-hydroxylase (CYP27) deficiency or mutation. The disorders associated with CYP27 deficiency include but not limited to cerebrotendinous xanthomatosis, cataracts, gallstone, tendon xanthomas, atherosclerosis, hepatomegaly, hypertriglyceridemia, and neurological and neuropsychiatric abnormalities such as peripheral neuropathy and dementia. In another preferred embodiment, PXR agonists are used to prevent or treat disorders that can be alleviated by enhancing the degradation of cholesterol or bile alcohols. The disorders that can be alleviated by enhancing the degradation of cholesterol or bile alcohols include, but not limited to, cardiovascular diseases, hypertension, atherosclerosis, dyslipidemia, obesity, hypercholesterolemia, hyperlipidemia, hyperlipoproteinemia, hyperchylomicronemia, hyperbetalipoproteinemia, dysbetalipoproteinemia, hyperprebetalipoproteinemia, mixed hyperlipidemia, cholestasis, cholesterolosis, gallstone, cataracts, and hepatomegaly.

### RELATED APPLICATION

This application claims the benefit of U.S. Provisional Application No. 60/371,701, which was filed on Apr. 12, 2002, which is hereby incorporated by reference in its entirety including drawings as fully set forth herein.

### FIELD OF THE INVENTION

The present invention relates to methods of preventing or treating disorders associated with the degradation of cholesterol and bile alcohols including cerebrotendinous xanthomatosis.

### BACKGROUND OF THE INVENTION

Cerebrotendinous Xanthomatosis (CTX) is an autosomal recessive, lipid metabolic disorder characterized by progressive deposition of cholesterol and cholestanol in many tissues, especially in eye lenses, central nervous systems and muscle tendons. Clinical manifestations of CTX include premature bilateral cataracts, tendon xanthomas particularly of the Achilles tendon, premature atherosclerosis, gallstone, and neurological and neuropsychiatric abnormalities such as pyradimal/cerebellar signs, peripheral neuropathy, and dementia.

It is reported that CTX results from mutations within the gene encoding sterol 27-hydroxylase (CYP27), a member of the mitochondrial cytochrome P-450 enzyme family involved in bile acid biosynthetic pathways. Andersson et al., Cloning, Structure, and Expression of the Mitochondrial CytochromeP-450 Sterol 27-Hydroxylase, A Bile Acid Biosynthetic Enzyme, J. Biol. Chem. 264: 8222-8229 (1989). Cali et al., Mutations in the Bile Acid Biosynthetic Enzyme Sterol 27-Hydroxylase Underlie Cerebrotendinous Xanthomatosis, J. Biol. Chem. 266: 7779-7783 (1991).

Bile acids are a group of sterol-derived compounds that act as detergents in the intestine to facilitate the digestion and absorption of fats and fat-soluble molecules. Bile acids are biosynthesized from cholesterol through a classical biosynthetic pathway and an alternate pathway as shown in FIG. 1.

The classical bile acid biosynthetic pathway, located in the endoplasmic reticulum of liver cells, starts with a-hydroxylation of carbon 7 of the cholesterol steroid nucleus which is catalyzed by a mitochondrial cytochrome P-450 monooxygenase, commonly known as cholesterol 7α-hydroxylase (CYP7A). 7α-hydroxycholesterol is converted to 7α-hydroxy-4-cholesten-3-one which undergoes subsequent enzymatic modifications and yields 5β-cholestane-3α, 7α-diol and 5β-cholestane-3α, 7α, 12α-triol. 5β-cholestane-3α, 7α, 12α-triol can be hydroxylated by CYP27 to form 5β-cholestane-3α, 7α, 12α, 27-tetrol which are finally converted into cholic acid. Alternatively, the 5β-cholestane-3α, 7α, 12α-triol is hydroxylated by a cytochrome P450 monooxygenase (CYP3A) to form 5β-cholestane-3α, 7α, 12α, 25-tetrol and then to form 5β-cholestane-3α, 7α, 12α, 24S, 25-pentol which is metabolized to cholic acid by cytosolic enzymes. Honda et al., Differences in hepatic levels of intermediates in bile acid biosynthesis between Cyp27−/−mice and CTX, J. Lip. Res. 42: 291-300 (2001).

The alternate bile acid biosynthetic pathway is present not only in liver cells but in extrahepatic organs as well, especially in the lungs. In the alternate pathway, cholesterol is hydroxylated by CYP27 to form oxysterols which eventually turn into chenodeoxycholic acid.

It is now well documented that CYP27 plays important roles in both bile acid synthetic pathways, particularly in the degradation of the steroid side chain in the conversion of cholesterol into bile acids. Indeed, the deficiency of CYP27 in humans results in marked reduction in bile acid synthesis, particularly by decreasing the formation of chenodeoxycholic acid. Due to the reduced formation of the bile acids, the negative feedback of the CYP7A is reduced and results in an up-regulation of CYP7A. Consequently, a large amount of 25-hydroxylated C27-bile alcohols including 5β-cholestane-3α, 7α, 12α, 25-tetrol, 5β-cholestane-3α, 7α, 12α, 24S, 25-pentol, and 5β-cholestane-3α, 7α, 12α, 24R, 25-pentol, are accumulated and excreted in bile, feces and urine. CYP27 deficiency also leads to the accumulation and deposit of cholestanol in tissues which results from the conversion of 7α-hydroxy-4-cholesten-3-one into cholestanol by hepatic enzymes.

The accumulation of bile alcohols in serum and urine and the deposit of cholestanol in tissues of CYP27 deficient patients are consistent with the clinical manifestations of CTX. Interestingly, however, CYP27 knockout mice fail to show typical CTX-related biochemical features. Rosen et al., Markedly Reduced Bile Acid Synthesis but Maintained Levels of Cholesterol and Vitamin D Metabolites in Mice with Disrupted Sterol 27-Hydroxylase Gene, J. Biol. Chem. 273: 14805-14812 (1998). It has been found that microsomal 25-and 26-hydroxylations of 5β-cholestane-3α, 7α, 12α-triol and microsomal 23R-, 24R-, 24S-, and 27-hydroxylations of 5β-cholestane-3α, 7α, 12α, 25-tetrol are mainly catalyzed by CYP3A in both human and mice. It has been observed that CYP7A is not up-regulated but CYP3A activity is up-regulated in CYP27 −/− mice. In contrast, considerable up-regulation of CYP7A without elevation of CYP3A is observed in CTX humans. It has therefore been hypothesized that the elevated activity of CYP3A in mice but not in humans provides a salvage pathway for the hydroxylations of bile alcohols rather than resulting in pathological features of CTX. Honda et al., Side Chain Hydroxylations in Bile Acid Biosynthesis Catalyzed by CYP3A Are Markedly Up-Regulated in Cyp27 −/− Mice but Not in Cerebrotendinous Xanthomatosis, J. Biol. Chem. 276: 34579-34585 (2001)

The elevation of CYP3A activities in CYP27 −/− mice seems interesting, since CYP3A is not only involved in the bile acid biosynthetic pathway but also responsible for metabolism of about 60% of all clinically used drugs. In general, CYP3A substrates are large (Mr>300) lipophilic molecules that include antimycotics, macrolide antibiotics, contraceptive steroids, antiviral agents, and calcium channel blocker, to list a few. Michalets L., Update: Clinically Significant Cytochrome P-450 Drug Interactions, Pharmacotherapy 18: 84-112 (1998). CYP3A expression can be induced by dexamethasone, RU486, spironolactone, cyproterone acetate, the antifungal agent clotrimazole, the anticonvulsant phenytoin, the nonsteroidal antiinflammatory drug phenylbutazone, the proton pump inhibitors omeprazole and lansoprazole, and the anticancer agent paclitaxel. See, Jones et al., The Pregnane X Receptor: A Promiscuous Xenobiotic Receptor That Has Diverged During Evolution Mol. Endocrinol. 14: 27-39 (2000).

The deficiency of CYP27 gene also has a significant impact on hepatic fatty acid/triacylglycerol metabolism and adrenal cholesterol homeostasis. It has been reported that CYP27 disruption causes hypertriglyceridemia and hepatomegaly in mice. Repa et al., Disruption of the Sterol 27-Hydroxylase Gene in Mice Results in Hepatomegaly and Hypertriglyceridemia, J. Biol. Chem. 275: 39685-39692 (2000).

Currently, most of CTX patients with CYP27 deficiency are treated with chenodeoxycholic acid. It has been reported that long-term therapy with chenodeoxycholic acid in CTX may correct the biochemical abnormalities of CTX. Berginer et al., Long-Tern Treatment of Cerebrotendinous Xanthomatosis with Chenodeoxycholic Acid, N. Eng. J. Med. 27: 1649-1652 (1984). However, the chenodeoxycholic acid therapy is accompanied by major adverse effects such as diarrhea, restlessness and impatience. In some cases, CTX patients are treated with statins. However, the use of statins is controversial since there is a possibility of worsening the CTX condition owing to increased low-density lipoprotein uptake as the result of augmented low-density lipoprotein receptor activity. Surgical removal of the Achilles tendon xanthomas is also considered, yet may worsen the gait in neurologically affected patients.

Therefore, novel treatments for CYP27 deficient humans through efficient degradation of cholesterol and bile alcohols by perfecting or improving bile acid biosynthetic pathways would be highly desirable. In line with the enhanced bile acid biosynthetic pathways, there is a need for methods to prevent or treat disorders which can be alleviated through reducing cholesterol or enhancing the bile acid biosynthetic pathway. The disorders which can be alleviated through perfecting or improving the bile acid biosynthetic pathways include hyperlipidemia, hypertriglyceridemia, dyslipidemia, hypertension, cardiovascular diseases, and obesity. Finally, it would be desirable to develop methods to reduce drug toxicity or increase drug efficacy or pharmacodynamics through the regulation of the activity of enzymes, e.g., CYP3A, which are involved in the metabolism of bile alcohols as well as drug clearance.

### SUMMARY OF THE INVENTION

The primary aspect of the present invention is directed to the treatment of a disorder associated with CYP 27 deficiency in humans by administering to a CYP27 deficient human a pharmaceutically effective dose of a human PXR agonist or a human PXR agonist composition. In one embodiment of the invention, the disorder associated with CYP27 deficiency includes cerebrotendinous xanthomatosis (CTX), cataracts, gallstone, tendon xanthomas, atherosclerosis, hepatomegaly, hypertriglyceridemia, and neurological and neuropsychiatric abnormalities such as peripheral neuropathy and dementia. In another embodiment of the invention, the human PXR agonist is selected from the group consisting of dexamethasone t-butylacetate, 11β-(4-dimethylaminophenyl)-17β-hydroxy-17α-propinyl-4, 9-estradiene-3-one (RU486, Mifepristone), corticosterone, rifampicin, nifedipine, clotrimazole, bisphosphonate ester SR12813, hyperforin (a component of St. John's wort), paclitaxel (Taxol), ritonavir, lithocholic acid, and 3-keto-lithocholic acid.

Another aspect of the present invention is directed to the treatment or prevention of a disorder in a subject that can be alleviated through enhanced degradation of cholesterol or bile alcohols by administering to the subject a pharmaceutically effective dose of a PXR agonist. In one embodiment of the invention, the disorder that can be alleviated through enhanced degradation of cholesterol or bile alcohols includes cerebrotendinous xanthomatosis, cardiovascular diseases, hypertension, atherosclerosis, dyslipidemia, obesity, hypercholesterolemia, hyperlipidemia, hyperlipoproteinemia, hyperchylomicronemia, hyperbetalipoproteinemia, dysbetalipoproteinemia, hyperprebetalipoproteinemia, mixed hyperlipidemia, cholestasis, cholesterolosis, gallstone, cataracts, and hepatomegaly.

Another aspect of the present invention provides a method for treating or preventing a condition in a subject that can be alleviated by decreasing or inhibiting the degradation of cholesterol or bile alcohols by administering a pharmaceutically effective dose of a PXR antagonist or a PXR antagonist composition to the subject. Conditions that can be alleviated by decreasing or inhibiting the degradation of cholesterol or bile alcohols include those disorders that have reduced levels of lipoprotein. Such conditions include hypolipoproteinemia, hypobetalipoproteinemia, and abetalipoproteinemia.

Another aspect of the present invention is directed to enhancing the degradation of 5β-cholestane-3α, 7α, 12α-triol in the bile acid biosynthetic pathway by using a PXR agonist or a PXR agonist composition to activate a PXR. A preferred embodiment of the invention is directed to the use of a human PXR agonist or a human PXR agonist composition to activate a human PXR to enhance the degradation of 5β-cholestane-3α, 7α, 12α-triol.

Another aspect of the present invention is directed to drug metabolism. One embodiment of the invention is directed to a method for increasing efficacy and pharmacokinetics of a drug or reducing the CYP3A mediated clearance of the drug by administering to a subject a pharmaceutically effective dose of a PXR antagonist or a PXR antagonist composition. Another embodiment of the invention is directed to a method of decreasing the toxicity of a drug or improving the clearance of the drug by administering to a subject a pharmaceutically effective dose of a PXR agonist or a PXR agonist composition.

### DESCRIPTION OF THE DRAWINGS

FIG. 1 shows the bile acid biosynthetic pathway. Bile acids are synthesized from cholesterol via two different pathways, the classical (left side) and the alternative (right side). CYP27 catalyzes the indicated reactions in both pathways. The bile alcohols 5β-cholestane-3α, 7α, 12α-triol and 5β-cholestane-3α, 7α, 12α, 25-tetrol are normally metabolized by either CYP27 or CYP3A (as indicated), leading to the formation of the primary bile acid cholic acid (CA). 5β-cholestane-3α, 7α, 12α-triol is elevated in both CYP27 deficient human and mice. In CYP27 −/− mice, the elevated levels of 5β-cholestane-3α, 7α, 12α-triol activate mouse PXR which in turn stimulates CYP3A transcription. The enhanced activity of CYP3A metabolizes and eliminates 5β-cholestane-3α, 7α, 12α-triol and shunts the bile acid biosynthetic pathway into the formation of cholic acid through CYP3A mediated degradation of 5β-cholestane-3α, 7α, 12α-triol. However, 5β-cholestane-3α, 7α, 12α-triol cannot activate human PXR and therefore fails to stimulate CYP3A activity. Unlike their mouse counterparts, CYP27 deficient humans are incapable of catalyzing and eliminating 5β-cholestane-3α, 7α, 12α-triol. Instead, bile alcohols accumulate as a result of the inactivation of CYP27 and CYP3A which eventually lead to the clinical manifestations of CTX in CYP27 deficient humans.

FIG. 2 shows the chemical structure of selected human PXR agonists including Rifampicin, Taxol, SR12813, Hyperforin, and Ritonavir.

FIG. 3 shows that 5β-cholestane-3α, 7α, 12α-triol (Triol) and 5β-cholestane-3α, 7α, 12α, 25-tetrol (Tetrol) are endogenous sterols that activate mouse PXR. FIG. 3(a) shows that 5β-cholestane-3α, 7α, 12α-triol (Triol) and 5β-cholestane-3α, 7α, 12α, 25-tetrol (Tetrol) activate mouse PXR in a reporter gene assay. CV-1 cells were transiently transfected with a GAL-mouse PXR expression vector, a GAL4 reporter construct, and a β-galactosidase vector as an internal control. Where noted, the bile acid transporter NTCP was added to the transfection to promote the transport of membrane-impermeable bile acids. Transfected cells were exposed to the indicated compounds (10 μM each), and fold activation was determined using luciferase and β-galactosidase enzyme activity assays. PCN refers to pregnenolone-16α-carbonitrile which is an effective agonist to mouse PXR. FIG. 3(b) shows the same as FIG. 3(a) except that a full-length mouse PXR expression vector was used along with a reporter construct containing the transcriptional regulatory region of rat cyp3a2 that responds to the DNA binding domain of the mouse PXR. FIG. 3(c) shows a dose response analysis of 5β-cholestane-3α, 7α, 12α-triol (Triol) and 5β-cholestane-3α, 7α, 12α, 25-tetrol (Tetrol) activity. The transfected CV-1 cells were treated with multiple concentrations of each sterol. Error bars in this figure and subsequent figures represent the standard error of the mean (SEM) from representative experiments. In some cases, the error bars are not visible because they are negligible relative to the scale of the figure

FIG. 4 shows that 5β-cholestane-3α, 7α, 12α-triol (Triol) and 5β-cholestane-3α, 7α, 12α, 25-tetrol (Tetrol) interact with or bind to human PXR directly in an in vitro ligand displacement assay. Bacterially expressed human PXR ligand binding domain was incubated with [³H]SR12813 in the absence or presence of the following unlabeled competitors: 5 μM Hyperforin, 30 μM 5β-cholestane-3α, 7α, 12α-triol (Triol), and 30 μM 5β-cholestane-3α, 7α, 12α, 25-tetrol (Tetrol). The amount of [³H]SR12813 associated with human PXR is expressed in cpm. This figure shows that 5β-cholestan-3α, 7α, 12α-triol is equally as effective as Hyperforin in competing with [³H]SR12813 However, 5β-cholestane-3α, 7α, 12α, 25-tetrol is less effective.

FIG. 5 shows that 5β-cholestane-3α, 7α, 12α-triol (Triol) and 5β-cholestane-3α, 7α, 12α, 25-tetrol (Tetrol) modulate or activate the expression of endogenous mouse PXR target genes (cyp3a11, cyp2c, and oatp2). Primary mouse hepatocytes were treated with compounds (10 μM), and Northern analysis was performed using the probes as described in Example III.

FIG. 6 shows the properties of the liver extract of CYP27 null mice (CYP27 −/−) in comparison with wild-type mice. FIG. 6(a) shows elevated hepatic 5β-cholestane-3α, 7α, 12α-triol levels in the liver extract of CYP27 −/− mice. A gas chromatography-mass spectroscopy approach was used to measure 5-cholestane-3α, 7α, 12α-triol levels in liver extracts from wild type and CYP27-null mice (CYP27 −/−). FIG. (b) shows that an extract from CYP27-null liver activates mouse PXR. CV-1 cells were transfected with GAL-mouse PXR as described in Example I. Cells were then treated with equal amounts of organic extracts derived from the liver of wild type or cyp27-null mice. FIG. (c) shows that PXR target genes are induced in the liver of female cyp27-null mice. Northern analysis was performed as described in Example III.

FIG. 7 shows that CYP27-null (CYP27 −/−)mice are resistant to a xenobiotic challenge. Wild type and cyp27-null mice received an i.p. injection of tribromoethanol, and their individual sleep times are plotted. Wild type mice are represented by squares and CYP27-null mice (CYP27 −/−) are represented by circles. Wild type male, n=6; cyp27-1-male, n=5; Wild type female, n=6; cyp27-1-female, n=6. **, P<0.01; ***, P<0.001.

FIG. 8 shows that 5β-cholestane-3α, 7α, 12α-triol (Triol) and 5β-cholestane-3α, 7α, 12α, 25-tetrol (Tetrol) do not activate human PXR. FIG. 8(a) shows that 5β-cholestane-3α, 7α, 12α-triol is a weak agonist of human PXR. CV-1 cells were transfected as described in Example I with either GAL-mouse PXR (Gal-mouse PXR, Left) or GAL-human PXR (Gal-human PXR, Right). Ligands were as follows: 2.5 μM hyperforin and 10 μM pregnenolone-16α-carbonitrile (PCN), 5β-cholestane-3α, 7α, 12α-triol (Triol), 5β-cholestane-3α, 7α, 12α, 25-tetrol (Tetrol), and 7α, 12α-dihydroxy-4-cholesten-3-one. For mouse PXR, the data are plotted as percent of maximal foldactivation achieved with pregnenolone-16α-carbonitrile (PCN). For human PXR, the data are plotted as percent of maximal fold-activation achieved with hyperforin. FIG. 8(b) shows that 5β-cholestane-3α, 7α, 12α-triol (Triol) is a partial agonist/antagonist of human PXR. Experimental conditions were as in FIG. 8(a) using human PXR. FIG. 8(c) shows that 5β-cholestane-3α, 7α, 12α-triol (Triol) fails to activate human CYP3A4 expression. Northern analysis was performed as described in Example I but using primary human hepatocytes and the following ligands: 2.5 μM hyperforin and 10 μM rifampicin, 5β-cholestane-3α, 7α, 12α-triol (Triol), and 5β-cholestane-3α, 7α, 12α, 25-tetrol (Tetrol).

### DETAILED DESCRIPTION OF THE INVENTION

The present invention relates to an unexpected finding that 5β-cholestan-3α, 7α, 12α-triol, a CYP3A substrate and bile alcohol, is an effective endogenous agonist to mouse pregnane X receptors (PXR) which regulate the induction and expression of CYP3A; however, 5β-cholestane-3α, 7α, 12α-triol fails to induce human PXR.

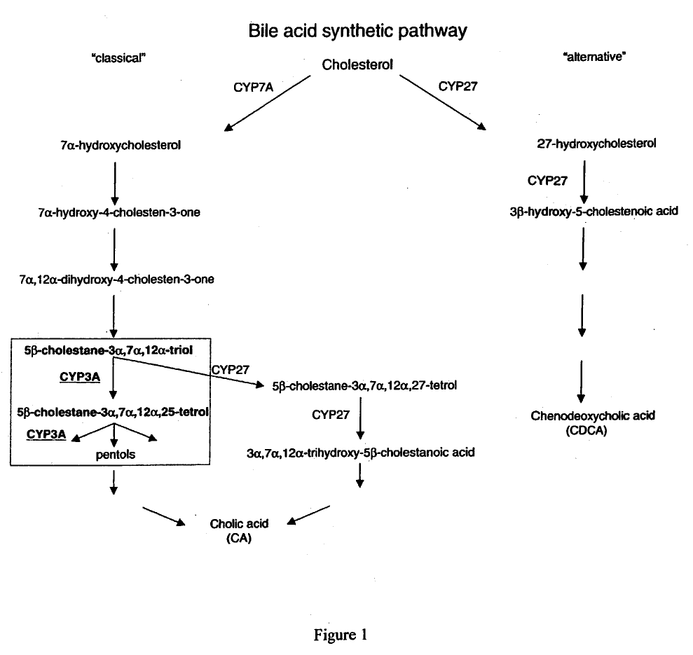

Without being limited to any theory, the unexpected finding provides an explanation to the difference in CYP3A activity and clinical manifestations between CYP27 −/− mice and CYP27 deficient humans. In both CYP27 −/− mice and CYP27 deficient humans, the lack of CYP27 activity or the deficiency of CYP27 activity leads to the accumulation of 5β-cholestane-3α, 7α, 12α-triol which is a metabolic intermediate or bile alcohol in the bile acid biosynthetic pathway. The bile acid biosynthetic pathway is shown in FIG. 1. In CYP27 −/− mice, however, the accumulated 5β-cholestane-3α, 7α, 12α-triol reaches to an amount sufficient to activate endogenous mouse PXR. The activation of mouse PXR induces PXR target gene CYP3A which then metabolizes 5β-cholestane-3α, 7α, 12α-triol into 5β-cholestane-3α, 7α, 12α, 25-tetrol and 5β-cholestane-3α, 7α, 12α, 24S, 25-pentol both of which are eventually converted into cholic acid. Consequently, even in the absence of CYP27 activity, CYP27 −/− mice do not accumulate 5β-cholestane-3α, 7α, 12α-triol, 5β-cholestane-3α, 7α, 12α, 25-tetrol, or 5β-cholestan-3α, and 7α, 12α, 24S, 25-pentol, nor do CYP27 −/− mice develop clinical abnormalities as observed in CYP27 deficient humans.

In marked contrast, the accumulation of endogenous 5β-cholestane-3α, 7α, 12α-triol does not induce human PXR or CYP3A. This prevents bile alcohols in CYP27 deficient humans from undergoing a CYP3A mediated degradation pathway to eliminate 5β-cholestane-3α, 7α, 12α-triol due to the lack of up-regulation in CYP3A activity. Consequently, 5β-cholestane-3α, 7α, 12α-triol and 5β-cholestane-3α, 7α, 12α, 25-tetrol accumulate, resulting in biochemical abnormalities of CTX.

Therefore, the present invention is directed to the use of a PXR agonist to human PXR or a human PXR agonist to activate human PXR which then induces the up-regulation of CYP3A for 1) the CYP3A mediated degradation of 5β-cholestane-3α, 7α, 12α-triol into cholic acid; 2) the treatment of disorders or conditions associated with CYP27 deficiency; 3) the prevention or treatment of disorders or conditions associated with the degradation of cholesterol and bile alcohols that can be alleviated through the regulation of CYP3A activity; and 4) drug metabolism involved in CYP3A mediated clearance.

Since a PXR agonist induces the activity of a PXR which in turn up-regulates the expression of CYP3A, 5β-cholestane-3α, 7α, 12α-triol is expected to be degraded into cholic acid via a CYP3A mediated pathway for bile alcohols in the presence of the PXR agonist. Therefore, one aspect of the present invention provides a method for increasing the degradation of 5β-cholestane-3α, 7α, 12α-triol in a cell by contacting the cell with a PXR agonist. The cell can be a cell that is involved in the degradation of 5β-cholestane-3α, 7α, 12α-triol. In a preferred embodiment, the cell is a hepatic cell. The cell can be a CYP27 −/−cell or a CYP27 +/+ cell. The cell can be a cell of mammalian origin. It is preferred that the cell is a human cell. It is most preferred that the cell is a human hepatic cell. The advantage of this method is to enhance the degradation of cholesterol and 5β-cholestane-3α, 7α, 12α-triol and reduce the levels of cholesterol and bile alcohols in the bile acid biosynthetic pathway.

The diminished responsiveness of human PXR to endogenous bile alcohols, e.g., 5β-cholestane-3α, 7α, 12α-triol, defines a molecular mechanism that prevents CYP27 deficient humans from disposing of accumulative 5β-cholestane-3α, 7α, 12α-triol, since, unlike in CYP27 −/− mice, CYP3A mediated pathway is not induced in CYP27 deficient humans to convert 5β-cholestane-3α, 7α, 12α-triol into cholic acid. This provides a rationale that the CYP3A mediated pathway in CYP27 deficient humans may be activated when a human PXR is induced by a human PXR agonist. In the presence of a human PXR agonist, the CYP3A may be up-regulated and metabolize the accumulated 5β-cholestane-3α, 7α, 12α-triol and lead to the degradation of bile alcohols into cholic acids. Consequently, the biochemical abnormalities of CTX or disorders associated with CYP27 deficiency may be reduced, corrected, or prevented. Therefore, another aspect of the present invention provides a method for treating disorders associated with CYP27 deficiency in a human by administering a pharmaceutically effective dose of a human PXR agonist or a human PXR agonist composition to the human. The disorders associated with CY27 deficiency are pathological abnormalities or symptoms commonly observed in CYP27 mutant or deficient humans which include cerebrotendinous xanthomatosis, cataracts, gallstone, tendon xanthomas particularly of Achilles tendon, atherosclerosis, hepatomegaly, hypertriglyceridemia, and neurological and neuropsychiatric abnormalities such as pyradimal and cerebellar signs, peripheral neuropathy, and dementia. The genetic characteristics of CYP27 deficiency or mutations are well defined in the art. Verrips et al., Clinical and Molecular Genetic Characteristic of Patients with Cerebrotendinous Xanthomatosis, Brain: 123, 908-919 (2000).

As a PXR agonist activates a PXR which in turn up-regulates the activity of CYP3A, the elevated CYP3A would be expected to enhance the degradation of cholesterol and bile alcohols and reduce cholesterol levels. Accordingly, another aspect of the present invention provides a method for enhancing or facilitating the degradation of cholesterol or bile alcohols in a subject in need thereof by administering to the subject a pharmaceutically effective dose of a PXR agonist or a PXR agonist composition. The subject in need thereof is a subject which has a condition that can be alleviated by enhancing or facilitating the degradation of cholesterol or bile alcohols. In a preferred embodiment, another aspect of the present invention provides a method for preventing or treating a condition in a subject that can be alleviated by enhancing or facilitating the degradation of cholesterol or bile alcohols by administering a pharmaceutically effective dose of a PXR agonist or a PXR agonist composition to the subject. The PXR agonist may also be used to reduce cholesterol levels, particularly levels of low-density lipoprotein cholesterol (LDL). The PXR agonist may further be used to improve the ratio between low-density lipoprotein cholesterol and high-density lipoprotein (HDL) cholesterol by lowering the levels of LDL or elevating the levels of HDL. The disorder that can be alleviated by enhancing or facilitating the degradation of cholesterol or bile alcohol refers to any condition that is caused by, complicated by or aggravated by an accumulation of cholesterol or bile alcohols and that can be reduced or lessened by the reduction of cholesterol or bile alcohol through the enhanced degradation in bile acid biosynthetic pathways. Such conditions include cerebrotendinous xanthomatosis, cardiovascular diseases, hypertension, atherosclerosis, dyslipidemia, obesity, hypercholesterolemia, hyperlipidemia, hyperlipoproteinemia, hyperchylomicronemia, hyperbetalipoproteinemia, dysbetalipoproteinemia, hyperprebetalipoproteinemia, mixed hyperlipidemia, cholestasis, cholesterolosis, gallstone, cataracts, and hepatomegaly. In a preferred embodiment, the subject is a human; the PXR agonist is a human PXR agonist, and the PXR agonist composition is a human PXR agonist composition.

Conversely, since a PXR antagonist inhibits the activity of a PXR as well as that of CYP3A, another aspect of the present invention provides a method for treating or preventing a disorder in a subject that can be alleviated by decreasing or inhibiting the degradation of cholesterol or bile alcohols by administering a pharmaceutically effective dose of a PXR antagonist or a PXR antagonist composition to the subject. Disorders that can be alleviated by decreasing or inhibiting the degradation of cholesterol or bile alcohol refer to disorders that have reduced level of lipoprotein. Such conditions include hypolipoproteinemia, hypobetalipoproteinemia, and abetalipoproteinemia. In a preferred embodiment, the subject is human, the PXR antagonist is a human PXR antagonist, and the PXR antagonist composition is a human PXR antagonist composition.

It is reported that altering the activity of PXR would modulate drug clearance. Synold et al., Methods of Modulating Drug Clearance Mechanisms by Altering SXR Activity, U.S. patent application Ser. No. 09/815,300, filed Feb. 21, 2002. It is found that a PXR ligand would alter the activity of a PXR which in turn modulates the activity of CYP3A which is responsible for the clearance of about 60% of all clinically used drugs. Xie & Evans, Orphan Nuclear Receptors: The Exotics of Xenobiotics, J. Biol. Chem. 276: 37739-37742(2001). Accordingly, another aspect of the present invention provides a method for improving clearance of a drug or reducing toxicity of a drug by administering a pharmaceutically effective dose of a PXR agonist or a PXR agonist composition to the subject. Likewise, another aspect of the present invention provides a method for increasing the efficacy or pharmacokinetics of a drug by administering a pharmaceutically effective dose of a PXR antagonist or a PXR antagonist composition to the subject. The “drug” as used herein are those clinically used drugs that are subject to CYP3A mediated metabolic clearance, which include but are not limited to HIV protease inhibitors, Tamoxifen, trans-retinoic acid, Tolbutamide, Atovastatin, Gemfibrozol, Amiodarone, Anastrozole, Azithromycin, Cannabinoids, Cimetidine, Clarithromycin, Clotrimazole, Cyclosporine, Danazol, Delavirdine, Dexamethasone, Diethyldithiocarbamate, Diltiazem, Dirithromycin, Disulfiram, Entacapone, Erythromycin, Ethinyl estradiol, Fluconazole, Fluoxetine, Fluvoxamine, Gestodene, Grapefruit juice, Indinavir, Isoniazid, Itraconazole, Ketoconazole, Metronidazole, Mibefradil, Miconazole, Nefazodone, Nelfinavir, Nevirapine, Norfloxacin, Norfluoxetine, Omeprazole, Oxiconazole, Paclitaxel (Taxol), Paroxetine, Propoxyphene, Quinidine, Quinine, Quinupristin, Dalfopristin, Ranitidine, Ritonavir, Saquinavir, Sertindole, Sertraline, Troglitazone, Troleandomycin, Valproic acid, Verapamil, Zafirlukast and Zileuton.

Pregnane X receptor (PXR) as used herein, also known as pregnane activated receptor (PAR) and steroid and xenobiotic receptor (SXR), is a member of the nuclear receptor superfamily including the steroid, retinoid and thyroid hormone receptors. DNA sequences encoding the full-length mouse, rat, rabbit, and human PXR have been cloned and sequenced. For example, the coding sequence for a human PXR is amino acid residues from #1 to #434 of GenBank accession number AF061056. The coding sequence for a mouse PXR is amino acid residues from #1 to #431 of GenBank accession number AF031814. See also, Bertilsson et al., Identification of a human nuclear receptor defines a newsignaling pathway for CYP3A induction, Proc. Natl. Acad. Sci. USA 95:12208-12213 (1998); Blumberg et al., SXR a novel steroid and xenobioticsensing nuclear receptor, Gene & Dev. 12: 3195-3205 (1998); Kliewer et al., An Orphan Nuclear Receptor Activated by Pregnanes Defines a Novel Steroid Signaling Pathway, Cell 92: 73-82 (1998); Lehmann et al., The Human Orphan Nuclear Receptor PXR Is Activated by Compounds That Regulate CYP3A4 Gene Expression and Cause Drug Interactions, J. Clin. Invest. 102:1016-1023 (1998).

Similar to other members of the nuclear receptor family, the polypeptide for PXR comprises a DNA binding domain (DBD) at the amino terminal region and a ligand binding domain (LBD) at the carboxyl terminal region. DBD binds to the regulatory region of PXR's target genes. LBD serves as an interacting site for PXR's ligand and also contains a transcriptional activation domain such as the activation function 2 (AF-2) helix. The binding of a PXR ligand to the LBD leads to a conformational change in the AF-2 helix and allows PXR to interact with accessory proteins and/or the transcriptional regulatory region of a PXR's target gene which is then activated if the ligand is a PXR agonist or deactivated if the ligand is a PXR antagonist. Bourguet et al., Nuclear receptor ligand-binding domains: three-dimensional structures, molecular interactions and pharmacological implications, Trends Pharmacol Sci. 21: 381-386 (2000).

On the other hand, PXR is different from other members of the nuclear receptor family in the following two aspects. First, the mouse and human receptors share only 76% amino acid identity in ligand-binding domains which represents a high degree of divergence for homologous members of the nuclear receptor family. See, . Jones et al., The Pregnane X Receptor: A Promiscuous Xenobiotic Receptor That Has Diverged During Evolution, Mol. Endocrinol. 14: 27-39 (2000). Second, although most nuclear receptors have evolved a high degree of ligand binding specificity, PXR is activated by a diverse array of PXR agonists.

PXR target genes are genes having transcriptional regulatory regions upstream from the transcription initiation site that interact with the DNA binding domain of PXR. The interaction of the transcriptional regulatory regions and the DNA binding domain of PXR in the presence of a PXR ligand would activate or repress the expression of the PXR target genes. The transcriptional regulatory region of PXR target genes usually shares a common feature. The sequence of the transcriptional regulatory region comprises a six base pair core sequence that are often organized as direct repeats (DR), everted repeats (ER), or inverted repeats (IR), separated by 0 to 8 nucleotides.

In a preferred embodiment, CYP3A is a target gene of PXR. Synold et al., The orphan nuclear receptor SXR coordinately regulates drug metabolism and efflux, Nature Med. 7:584-590 (2001). CYP3A23 (a rat CYP3A gene) has a regulatory region, bases −220 to −56 relative to the transcription initiation site, which contains three sites (sites A, B and C) having sequences known to be recognized by members of the nuclear receptor family. Site A (bases −110 to −91) is over 80% identical to the consensus binding site for the orphan nuclear receptor hepatocyte nuclear factor−4; site B (bases −136 to −118) comprises a DR of the AGTTCA motif separated by three nucleotides (DR3); and site C (bases −169 to −144) contains an imperfect everted repeat with a 6 nucleotide spacer (ER6) or a direct repeat with a 4-nucleotide spacer (DR4). It is reported that a mouse PXR binds to the DR3 in site B and the ER6 in site C of the regulatory region of CYP3A23. Kliewer et al., An Orphan Nuclear Receptor Activated by Pregnanes Defines a Novel Steroid Signaling Pathway, Cell 92: 73-82 (1998); Lehmann et al., The Human Orphan Nuclear Receptor PXR Is Activated by Compounds That Regulate CYP3A4 Gene Expression and Cause Drug Interactions, J. Clin. Invest. 102:1016-1023 (1998). When a PXR binds or interacts with a PXR ligand, the DBD of the PXR binds to the transcriptional regulatory region of CYP3A with or without accessory proteins and thus induces or inhibits the expression of CYP3A gene.

A PXR ligand as used herein refers to any molecule or compound which activates or repress a PXR, usually by interaction with the ligand binding domain of a PXR. However PXR ligand can also be a compound or molecule which activates or represses a PXR without binding.

To determine whether a molecule or compound interacts directly with a PXR or is a PXR ligand, an in vitro ligand displacement assay is commonly used. Dussault et al., Peptide Mimetic HIV Protease Inhibitors Are Ligands for the Orphan Receptor SXR, J. Biol. Chem. 276: 33309-33312 (2001). In the ligand displacement assay, the ligand binding domain of a PXR is synthesized or expressed and purified. A known PXR ligand is radio-labeled and incubated with the purified ligand binding domain of the PXR. A molecule or compound suspected to be a PXR ligand is then added into the incubated mixture. Radioactivity reading is taken using a scintillation counter. If the molecule or compound competes with the known PXR ligand for the ligand binding domain of the PXR, the molecule or compound is expected to displace some of the radio-labeled known PXR agonist and reduce the radioactivity reading. Accordingly, the reduction of radioactivity reading indicates whether the molecule or compound is a PXR ligand and the extent to which the molecule or compound competes with the known PXR ligand.

A PXR agonist refers to a PXR ligand that activates a PXR. A PXR agonist can be a PXR ligand that interacts with or binds to the ligand binding domain of a PXR. Once a PXR agonist interacts or binds to the ligand binding domain of a PXR, the PXR agonist—PXR complex undergoes a conformational change and causes a DNA binding domain to bind to a regulatory region of a PXR target gene with or without an accessory protein. Consequently, the PXR agonist causes the up-regulation or enhanced expression of a PXR target gene. The PXR agonist may further cause the up-regulation or enhanced activity of a target gene product. A PXR agonist can also be a PXR ligand that increases the interaction of a PXR with another molecule, e.g., the regulatory region of a PXR target gene or an accessory protein to PXR. An example of an accessory protein to PXR is a retinoic acid receptor. Lehmann et al., The Human Orphan Nuclear Receptor PXR Is Activated by Compounds That Regulate CYP3A4 Gene Expression and Cause Drug Interactions, J. Clin. Invest. 102:1016-1023 (1998). A PXR agonist can be xenobiotic or endogenous. Examples of endogenous PXR agonists include 5β-cholestane-3α, 7α, 12α-triol, 5β-cholestane-3α, 7α, 12α, 25-tetrol and lithocholic acid. Examples of xenobiotic PXR agonists include 11β-(4-dimethylaminophenyl)-17β-hyrdoxy-17α-propinyl-4, 9-estradiene-3-one (RU486, Mifepristone).

Since PXR receptors from various species share less homology in the gene sequence of the ligand binding domain than other nuclear receptor, a PXR agonist capable of interacting with a PXR from one species may or may not be able to induce a PXR of another species. As an example, 5β-cholestane-3α, 7α, 12α-triol is a mouse PXR agonist but not a human PXR agonist.

PXR agonists to mouse PXR or mouse PXR agonists include but are not limited to 5-alpha-pregnane-3, 20-dione, dexamethasone t-butylacetate, 11β-(4-dimethylaminophenyl)-17β-hyrdoxy-17α-propinyl-4, 9-estradiene-3-one (RU486, Mifepristone), corticosterone, pregnenolone-16α-carbonitrile (PCN), 5β-cholestane-3α, 7α, 12α-triol, 5β-cholestane-3α, 7α, 12α, 25-tetrol, lithocholic acid, 3-keto-lithocholic acid, trans-nonacholar and chlordane, polychlorinated biphenyls, antimineralocorticoid spironolactone, antiandrogen cyproterone acetate, nonylphenol and phthalic acid.

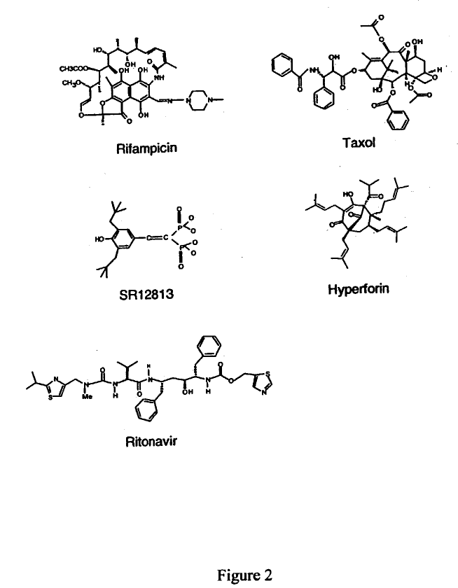

PXR agonists to human PXR or human PXR agonists include but are not limited to, dexamethasone t-butylacetate, 11β-(4-dimethylaminophenyl)-17β-hyrdoxy-17α-propinyl-4, 9-estradiene-3-one (RU486, Mifepristone), corticosterone, rifampicin, nifedipine, clotrimazole, bisphosphonate ester SR12813, hyperforin (a component of St. John's wort), paclitaxel (Taxol), ritonavir, lithocholic acid, and 3-keto-lithocholic acid. The chemical structure of selected human PXR agonists is shown in FIG. 2.

To determine whether or not a molecule or a compound is a PXR agonist, a reporter gene assay can be used. Blumberg et al., SXR, a novel steroid and xenobioticsensing nuclear receptor, Gene & Dev. 12: 3195-3205 (1998); Dussault et al., Peptide Mimetic HIV Protease Inhibitors Are Ligands for the Orphan Receptor SXR, J. Biol. Chem. 276: 33309-33312 (2001). In the reporter gene assay, eukaryotic cells are transiently co-transfected with a cluster of vectors which include an expression vector, a reporter plasmid and a control plasmid. The expression vector is used to express the ligand binding domain of a PXR and a DNA binding domain which can either be(the DNA binding domain of the PXR or a yeast Gal4 DNA binding domain. The reporter plasmid is constructed to include a reporter gene and a transcriptional regulatory sequence that interacts with the DNA binding domain of the PXR or yeast Gal4 DNA binding domain. Commonly used reporter genes are luciferase, beta-galactosidase and chloramphenicol acetultransferase (CAT). The control plasmid is engineered to have a control report gene which is selected from one of the reporter genes but not used in the reporter plasmid. The control plasmid does not contain the transcriptional regulatory sequence. The transiently transfected cell is then exposed to the molecule or compound and cell lysates are assayed for the appropriated reporter gene activity. Since these reporter genes encode bacterial enzymes which are either absent from non-transfected eukaryotic cells or present at very low levels, the presence and quantity of the enzymes can be monitored by simple and sensitive enzyme assays without interference from host cell enzymes. The enzyme assays are well known in the art and commercially available. See, Current Protocols in Molecular Biology. If the molecule or compound is indeed a PXR agonist, the molecule or compound would bind to the ligand binding domain of the PXR. This binding would cause the DNA binding domain to interact with the corresponding transcriptional regulatory sequence in the reporter plasmid and initiate the transcription and expression of the reporter gene. Consequently, the reporter enzyme assay would unveil the presence and level of the reporter gene when compared with the control report gene whose enzymatic activity is unaffected due to the lack of the transcriptional regulatory sequence. However, if the molecule or compound fails to interact with the ligand binding domain of the PXR, the enzymatic activity of the reporter gene would remain unaffected at the same base level as the control reporter gene.

Additionally, a Northern blot analysis of PXR target genes can be used to determine whether or not a molecule or compound is a PXR agonist. Dussault et al., Peptide Mimetic HIV Protease Inhibitors Are Ligands for the Orphan Receptor SXR, J. Biol. Chem. 276: 33309-33312 (2001). In this approach, hepatocytes are isolated, cultured in vitro, and exposed to a molecule or compound suspected to be a PXR agonist. Total RNA of the hepatocytes is isolated and subject to Northern blot analysis with probes of PXR target genes' fragments. The PXR target genes have transcriptional regulatory regions that interact with the DNA binding domain of a PXR. When a PXR agonist interacts or binds to the ligand binding domain of a PXR, the DNA binding domain of a PXR interacts with the transcriptional regulatory region of the target genes and activates the expression of the target genes. Consequently, an enhanced expression of target genes is observed in the Northern blot analysis. Commonly used target genes' fragments include cyp3a11, nucleotides from #1,065 to #1,569 of GenBank accession no. X60452; cyp2c, nucleotides from #787 to #1,193 of GenBank accession no. AK008580; and oatp2, nucleotides 2,124-2,486 of GenBank accession no. NM_{—}021471. gapdh, nucleotides from #590 to #1,089 of GenBank accession no. NM_{—}008094, is used as a control since gapdh is not regulated by the activation of a PXR.

A PXR antagonist is a PXR ligand that interacts with or binds to a PXR receptor and down-regulates (or suppresses or inhibits ) the activity of a PXR. In particular, a PXR antagonist can be a molecule or compound that reverses or decreases a PXR agonist induced activity of a PXR or a PXR agonist induced activation of a PXR target gene. A PXR antagonist can be a molecule or a compound that inhibits or decreases the binding of a PXR to, e.g., the regulatory region of a target gene or an accessory protein to PXR, and therefore repress the activation of a PXR target gene's expression. A PXR antagonist can be a molecule or a compound that down-regulates the expression of a target gene or the activity of a target gene product through the PXR antagonist's interaction with a PXR. As an example, Ecteinascidin-743 (ET-743), a marine-derived antineoplastic agent, is a potent PXR antagonist with a half-maximal inhibitory concentration (IC₅₀) of 3 nM in antagonizing the PXR-dependent activation by a PXR agonist SR12813. In addition, ET-743 completely inhibits the induction of CYP3A by SR12813.

Methods to determine whether or not a molecule is a PXR antagonist are also known in the art. See, Synold et al., The orphan nuclear receptor SXR coordinately regulates drug metabolism and efflux, Nature Med. 7: 584-590 (2001). Briefly, a molecule suspected to be a PXR antagonist is mixed with a known PXR agonist and the mixture is incubated with transfected cells in the reporter gene assay as described in the present invention. If the activity of the PXR in cells treated with the mixture is inhibited or reduced in comparison with that in cells treated only with the known PXR agonist, the molecule is expected to be a PXR antagonist.

The term “pharmaceutically effective dose” as used herein refers to the amount of, e.g., a PXR ligand, a PXR ligand composition, which is effective for producing a desired therapeutic effect or alleviating conditions associated with disorders in the bile acid biosynthetic pathway by enhancing or inhibiting the activity of CYP3A via the activation or deactivation of PXR. As known in the art of pharmacology, the precise amount of the pharmaceutically effective dose of a PXR ligand or a PXR ligand composition that will yield the most effective results in terms of efficacy of treatment in a given patient will depend upon the activity, pharmacokinetics, pharmacodynamics, and bioavailability of a particular PXR ligand, physiological condition of the subject (including age, sex, disease type and stage, general physical condition, responsiveness to a given dosage and type of medication), the nature of pharmaceutically acceptable carrier in a formulation, a route of administration, etc. However, the above guidelines can be used as the basis for fine-tuning the treatment, e. g., determining the optimum dose of administration, which will require no more than routine experimentation consisting of monitoring the subject and adjusting the dosage. Remington: The Science and Practice of Pharmacy (Gennaro ed. 20^{th} edition, Williams & Wilkins PA., USA) (2000).

While it is possible for a PXR ligand to be administered as a pure or substantially pure compound, it is preferable that the PXR ligand be administered as a PXR ligand composition in the form of pharmaceutical formulations or preparations suitable for a particular administration route. A PXR ligand composition comprises a PXR ligand and a pharmaceutically acceptable carrier. In the case that a PXR ligand is a PXR agonist, a PXR ligand composition is a PXR agonist composition which comprises a PXR agonist and a pharmaceutically acceptable carrier. Likewise, a PXR antagonist composition is a PXR ligand composition that comprises a PXR antagonist and a pharmaceutically acceptable carrier. Additionally, a human PXR ligand (agonist or antagonist) comprises a human PXR ligand (agonist or antagonist) and a pharmaceutically acceptable carrier.

The term “pharmaceutically acceptable carrier” as used herein means a pharmaceutically-acceptable material, composition or vehicle, such as a liquid or solid filler, diluent, excipient, solvent or encapsulating material, involved in carrying or transporting a PXR ligand from one tissue, organ, or portion of the body, to another tissue, organ, or portion of the body. Each carrier must be “pharmaceutically acceptable” in the sense of being compatible with the other ingredients, e.g., the PXR ligand, of the formulation and suitable for use in contact with the tissue or organ of humans and animals without excessive toxicity, irritation, allergic response, immunogenecity, or other problems or complications, commensurate with a reasonable benefit/risk ratio. Some examples of materials which can serve as pharmaceutically-acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations

A PXR ligand or a PXR ligand composition can be administered to a subject by any administration route known in the art, including without limitation, oral, enteral, nasal, topical, rectal, vaginal, aerosol, transmucosal, transdermal, ophthalmic, pulmonary, and/or parenteral administration. A parenteral administration refers to an administration route that typically relates to injection. A parental administration includes but not limited to intravenous, intramuscular, intraarterial, intraathecal, intracapsular, infraorbital, intra cardiac, intradermal, intraperitoneal, transtracheal, subcutaneous, subcuticular, intraarticular, subcapsular, subarachnoid, intraspinal, and/or intrasternal injection and/or infusion.

Typically, a PXR ligand or a PXR ligand composition is given to a subject in the form of formulations or preparations suitable for each administration route. The formulations useful in the methods of the present invention include one or more PXR ligands, one or more pharmaceutically acceptable carriers therefor, and optionally other therapeutic ingredients. The formulations may conveniently be presented in unit dosage form and may be prepared by any methods well known in the art of pharmacy. The amount of active ingredient which can be combined with a carrier material to produce a single dosage form will vary depending upon the subject being treated, the particular mode of administration. The amount of PXR ligand which can be combined with a carrier material to produce a pharmaceutically effective dose will generally be that amount of the PXR ligand which produces a therapeutic effect. Generally, out of one hundred percent, this amount will range from about 1 percent to about ninety-nine percent of the PXR ligand, preferably from about 5 percent to about 70 percent.

Methods of preparing these formulations or compositions include the step of bringing into association a PXR ligand with one or more pharmaceutically acceptable carrier and, optionally, one or more accessory ingredients. In general, the formulations are prepared by uniformly and intimately bringing into association a PXR ligand with liquid carriers, or finely divided solid carriers, or both, and then, if necessary, shaping the product.

Formulations suitable for oral administration may be in the form of capsules, cachets, pills, tablets, lozenges (using a flavored basis, usually sucrose and acacia or tragacanth), powders, granules, or as a solution or a suspension in an aqueous or non-aqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of a PXR ligand as an active ingredient. A compound may also be administered as a bolus, electuary, or paste.

In solid dosage forms for oral administration (e. g., capsules, tablets, pills, dragees, powders, granules and the like), the PXR ligand is mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate, (5) solution retarding agents, such as paraffin, (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered peptide or peptidomimetic moistened with an inert liquid diluent.

Tablets, and other solid dosage forms, such as dragees, capsules, pills and granules, may optionally be scored or prepared with coatings and shells, such as enteric coatings and other coatings well known in the pharmaceutical-formulating art. They may also be formulated so as to provide slow or controlled release of the PXR ligand therein using, for example, hydroxypropylmethyl cellulose in varying proportions to provide the desired release profile, other polymer matrices, liposomes and/or microspheres. They may be sterilized by, for example, filtration through a bacteria-retaining filter, or by incorporating sterilizing agents in the form of sterile solid compositions which can be dissolved in sterile water, or some other sterile injectable medium immediately before use. These compositions may also optionally contain opacifying agents and may be of a composition that they release the PXR ligand(s) only, or preferentially, in a certain portion of the gastrointestinal tract, optionally, in a delayed manner. Examples of embedding compositions which can be used include polymeric substances and waxes. The PXR ligand can also be in micro-encapsulated form, if appropriate, with one or more of the above-described excipients.

Liquid dosage forms for oral administration include pharmaceutically acceptable emulsions, microemulsions, solutions, suspensions, syrups and elixirs. In addition to the PXR ligand, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcoho, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, coloring, perfuming and preservative agents.

Suspensions, in addition to the PXR ligand, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing one or more PXR agonist with one or more suitable nonirritating excipients or carriers comprising, for example, cocoa butter, polyethylene glycol, a suppository wax or a salicylate, and which is solid at room temperature, but liquid at body temperature and, therefore, will melt in the rectum or vaginal cavity and release the active agent. Formulations which are suitable for vaginal administration also include pessaries, tampons, creams, gels, pastes, foams or spray formulations containing such carriers as are known in the art to be appropriate.

Formulations for the topical or transdermal administration of a PXR ligand or a PXR ligand composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required. The ointments, pastes, creams and gels may contain, in addition to the PXR ligand or the PXR ligand composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the PXR ligand or the PXR ligand composition, excipients such as lactose, talc, silicic acid, aluminum hydroxide, calcium silicates and polyamide powder, or mixtures of these substances. Sprays can additionally contain customary propellants, such as chlorofluorohydrocarbons and volatile unsubstituted hydrocarbons, such as butane and propane.

PXR ligands or PXR ligand compositions can be alternatively administered by aerosol. This is accomplished by preparing an aqueous aerosol, liposomal preparation or solid particles containing the PXR ligands. A nonaqueous (e.g., fluorocarbon propellant) suspension could be used. Sonic nebulizers can also be used. An aqueous aerosol is made by formulating an aqueous solution or suspension of the agent together with conventional pharmaceutically acceptable carriers and stabilizers. The carriers and stabilizers vary with the requirements of the particular compound, but typically include nonionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Transdermal patches can also be used to deliver PXR ligands or PXR ligand compositions to the body. Such formulations can be made by dissolving or dispersing the agent in the proper medium. Absorption enhancers can also be used to increase the flux of the peptidomimetic across the skin. The rate of such flux can be controlled by either providing a rate controlling membrane or dispersing the peptidomimetic in a polymer matrix or gel.

Ophthalmic formulations, eye ointments, powders, solutions and the like, are also contemplated as being within the scope of this invention.

Formulations suitable for parenteral administration comprise a PXR ligand or a PXR ligand composition in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, bacterostats, solutes which render the formulation isotonic with the blood of the intended recipient or suspending or thickening agents.

Examples of suitable aqueous and nonaqueous carriers which may be employed in the formulations suitable for parenteral administration include water, ethanol, polyols (e. g., such as glycerol, propylehe glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

Formulations suitable for parenteral administration may also contain adjuvants such as preservatives, wetting agents, emulsifying agents and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

In some cases, in order to prolong the effect of a PXR ligand, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution which, in turn, may depend upon crystal size and crystalline form. Alternatively, delayed absorption of a parenterally-administered formulation is accomplished by dissolving or suspending the PXR ligand or PXR ligand composition in an oil vehicle.

Injectable depot forms are made by forming microencapsule matrices of a PXR ligand or in biodegradable polymers such as polylactide-polyglycolide. Depending on the ratio of the PXr ligand to polymer, and the nature of the particular polymer employed, the rate of drug release can be controlled. Examples of other biodegradable polymers include poly (orthoesters) and poly (anhydrides). Depot injectable formulations are also prepared by entrapping the PXr ligand in liposomes or microemulsions which are compatible with body tissue.

All references cited herein are incorporated by reference in their entirety. The descriptions in the present invention are provided only as examples and should not be understood to be limiting on the claims. Based on the description, a person of ordinary skill in the art may make modifications and changes to the preferred embodiments, which does not depart from the scope of the present invention.

### EXAMPLE I

5β-cholestane-3α, 7α, 12α-triol into 5β-cholestane-3α, 7α, 12α, 25-tetrol are endogenous sterols that activate mouse PXR.

5β-cholestane-3α, 7α, 12α-triol and 5β-cholestane-3α, 7α, 12α, 25-tetrol are endogenous CYP3A substrates in both human and mouse liver. Furster & Wikvall, Identification of CYP3A4 as the Major Enzyme Responsible for 25-Hydroxylation of 5β-cholestane-3α, 7α, 12α-triol in Human Liver Microsomes, Biochim. Biophys. Acta 1437: 46-52 (1999); Honda et al., Differences in hepatic levels of intermediates in bile acid biosynthesis between Cyp27−/−mice and CTX, J. Lip. Res. 42: 291-300 (2001). To determine whether 5β-cholestane-3α, 7α, 12α-triol and 5β-cholestane-3α, 7α, 12α, 25-tetrol activate mouse PXR, transient expression experiment and luciferase reporter gene assay were performed as described. in Dussault et al., Peptide Mimetic HIV Protease Inhibitors Are Ligands for the Orphan Receptor SXR, J. Biol. Chem. 276: 33309-33312 (2001). Briefly, a cytomegalovirus expression vector was used to express Gal-PXR fusion proteins. Gal-PXR fusion proteins contained the ligand binding domain of a PXR (human PXR ligand binding domain: GenBank accession number AF061056, amino acid residues from #107 to #443; mouse PXR ligand binding domain: GenBank accession number AF031814, amino acid residues from #104 to #431) linked to a yeast Gal4 DNA binding domain (GenBank accession number X85976, amino acid residues #1 to #147). Reporter plasmids were constructed by synthesizing three copy response elements that bind to the Gal4 DNA biding domain and subcloned into the transcriptional regulatory region of luciferase reporter gene. β-galactosidase expression vector pCH110, used as internal control, was obtained from Amersham Pharmacia Biotech. CV-1 cells were plated in 96-well plates at a density of 20,000 cells per well and maintained in DMEM supplemented with 10% charcoal/dextran treated calf bovine serum. Transient transfections were performed using DOTAP reagent (Boehringer Mannheim) at a concentration of 5 μg/ml in DMEM and a transfection mix containing cytomegalovirus expression vectors, reporter plasmids and β-galactosidase expression vectors. Compounds were added the next day in DMEM containing 10% delipidated fetal bovine serum. After 18-24 hr incubation, the cells were lysed and luciferase and β-galactosidase enzyme assays performed as known in the art. Reporter gene expression was normalized to the b-galactosidase transfection control and expressed as relative light units per OD per minute of β-galactosidase activity or fold induction over solvent control.

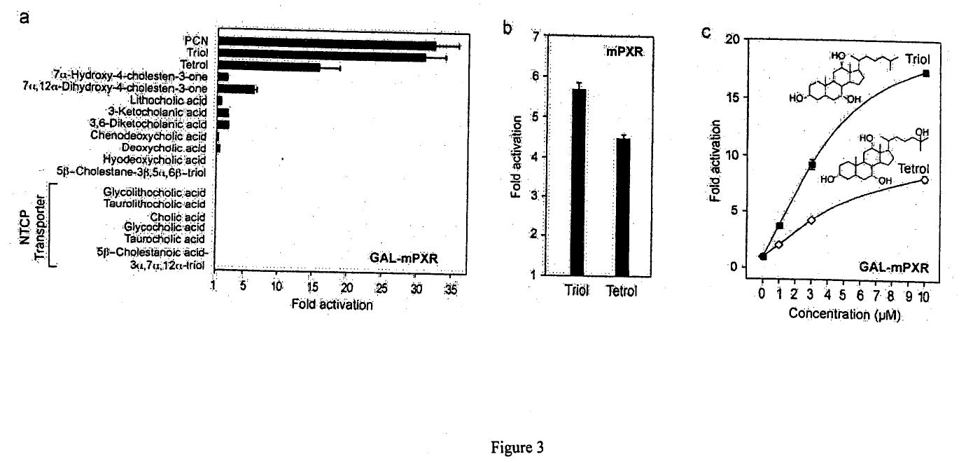

As shown in FIG. 3(a), pregnenolone-16α-carbonitrile (PCN) was a very effective agonist to mouse PXR at the concentration of 10 μM. The naturally occurring 5β-cholestane-3α, 7α, 12α-triol (Triol) was equally effective (31-fold) at the same concentration. On the other hand, 5β-cholestane-3α, 7α, 12α, 25-tetrol (Tetrol) also exhibited activity (16-fold) but was less efficacious than 5β-cholestane-3α, 7α, 12α-triol. Other cholesterol metabolites including lithocholic and 3-ketocholanic acids were inactive at 10 μM concentrations. These latter compounds were previously shown to activate PXR at higher concentrations, although they induce hepatic damage before activating PXR.

To further confirm that 5β-cholestane-3α, 7α, 12α-triol and 5β-cholestan-3α, 7α, 12α, 25-tetrol activate mouse PXR, the effect of these compounds on full-length mouse PXR was examined using a reporter construct containing a regulatory element from the rat cyp3a gene that binds to the DNA binding domain of mouse PXR. Blumberg et al., SXR, a novel steroid and xenobioticsensing nuclear receptor, Gene & Dev. 12: 3195-3205 (1998). As shown in FIG. 3(b), similar to the findings with GAL-mouse PXR, 10 μM 5β-cholestane-3α, 7α, 12α-triol (Triol) activated full-length mouse PXR, whereas 5β-cholestane-3α, 7α, 12α, 25-tetrol (Tetrol) also activated mouse PXR but was less effective. These findings show that 5β-cholestane-3α, 7α, 12α-triol and 5β-cholestane-3α, 7α, 12α, 25-tetrol are effective agonists to mouse PXR.

Dose response experiments indicated that triol activated mouse PXR with an approximate EC₅₀ of ≧3 μM (FIG. 3(c)). This concentration is close to the Michaelis constant reported for 5β-cholestane-3α, 7α, 12α-triol as a CYP3A4 substrate (Kₘ=6 μM). See, Furster & Wikvall, Identification of CYP3A4 as the Major Enzyme Responsible for 25-Hydroxylation of 5β-cholestane-3α, 7α, 12α-triol in Human Liver Microsomes, Biochim. Biophys. Acta 1437: 46-52 (1999). This finding indicated that 5β-cholestane-3α, 7α, 12α-triol (Triol) can associate with mouse PXR and CYP3A4 at similar concentrations. Since 5β-cholestane-3α, 7α, 12α-triol is an endogenous substrate for CYP3A4, the dose response experiments demonstrate that 5β-cholestane-3α, 7α, 12α-triol activates PXR at biologically relevant concentrations.

### EXAMPLE II

5β-cholestane-3α, 7α, 12α-triol and 5β-cholestane-3α, 7α, 12α, 25-tetrol interacts directly with human PXR.

An in vitro ligand displacement assay was used to determine whether 5β-cholestane-3α, 7α, 12α-triol interacts directly with the ligand binding domain of PXR. Because radiolabeled [³H]SR12813 are available for human PXR but not mouse PXR, human PXR was used to examine whether 5β-cholestane-3α, 7α, 12α-triol could compete for binding of human PXR to a tritiated SR12813.

The in vitro ligand displacement assay was described in Dussault et al., Peptide Mimetic HIV Protease Inhibitors Are Ligands for the Orphan Receptor SXR, J. Biol. Chem. 276: 33309-33312 (2001). Briefly, the human PXR ligand binding domain (GenBank accession number AF061056, amino acid residues from #1 to #107) was expressed in Escherichia coli with an N-terminal His tag and purified. For binding assays, 0.25 μg of His-tagged PXR was added per well of a 96-well nickel chelate flash plate (PerkinElmer Life Sciences) and incubated at room temperature for 30 min in binding buffer (50 mM Tris, pH 8, 50 mM KCl, 1 mM CHAPS, 0.1 mg/ml bovine serum albumin, and 0.1 mM dithiothreitol). After 30 min the well was washed three times with binding buffer, and 37.5 nM [³H]SR12813 was added in 100 μl of binding buffer. Unlabeled competitor compound were added, and the incubation was continued for 75 min at room temperature with shaking. Readings were taken using a Topcount scintillation counter (Packard, Meriden, Conn.).

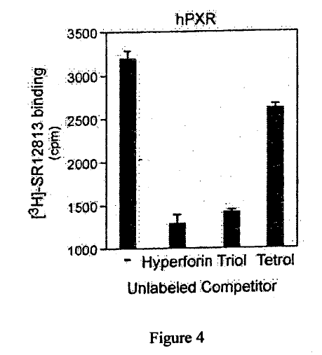

As shown in FIG. 4, human PXR bound to [³H]SR12813, and binding was effectively displaced by unlabeled hyperforin which is a high-affinity agonist to human PXR. Moore et al. (PNAS). The endogenous 5β-cholestane-3α, 7α, 12α-triol (Triol) was as equally effective as hyperforin in competing with [³H]SR12813. However, 5β-cholestane-3α, 7α, 12α, 25-tetrol (Tetrol) was less effective. A variety of related compounds that failed to activate mouse PXR as shown in FIG. 3(a) also failed to displace the radiolabeled SR12813. These inactive compounds include 7α-hydroxy-4-cholesten-3-one, 7α, 12α-dihydroxy-4-cholesten-3-one, 5β-cholestanoic acid-3α,7α,12α-triol, chenodeoxycholic acid, and cholic acid. Taken together, these findings demonstrate that 5β-cholestane-3α, 7α, 12α-triol and 5β-cholestane-3α, 7α, 12α, 25-tetrol directly interact with human PXR.

### EXAMPLE III

5β-cholestane-3α, 7α, 12α-triol and 5β-cholestane-3α, 7α, 12α, 25-tetrol modulate the expression of PXR target genes.

Mouse hepatocytes were isolated from C57BL6/J mice by using collagenase type IV. See, Bissell & Guzelian, Phenotypic Stability of Adult rat Hepatocytes in Primary Monolayer Culture, Ann. N.Y. Acad. Sci. 349: 85-98 (1980). 5×10⁵ cells per well were plated in six-well collagen-coated plates and cultured in DMEM/Ham's F12 media (1:1) containing 10 nM dexamethasone. Seventy hours after plating, the cells were treated with compounds for an additional 24 h. Total RNA was then isolated using Trizol reagent, and Northern blots were prepared with 10 μg of RNA per lane and probed with the following fragments: cyp3a11, nucleotides from #1,065 to #1,569 of GenBank accession no. X60452; cyp2c, nucleotides from #787 to #1,193 of GenBank accession no. AK008580; oatp2, nucleotides 2,124-2,486 of GenBank accession no. NM_{—}021471; and gapdh, nucleotides 590-1,089 of accession no. NM_{—}008094. cyp3a11, cyp2c, and oatp2 all have regulatory regions that interact with the DNA binding domain of PXR. However, gapdh is not regulated by the activation of PXR.

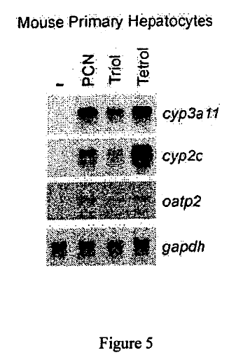

As shown in FIG. 5, the synthetic ligand PCN activated expression of the PXR target genes cyp3a11, cyp2c, and oatp2 but had no effect on the gapdh control. This finding corresponded well with previously results reported in the art. Synold et al., The orphan nuclear receptor SXR coordinately regulates drug metabolism and efflux, Nature Med. 7: 584-590 (2001); Xie et al., Humanized Xenobiotic Response in Mice Expressing Nuclear Receptor SXR, Nature 406: 435-439 (2000). It was also observed that 5β-cholestane-3α, 7α, 12α-triol (Triol) and 513-cholestane-3α, 7α, 12α, 25-tetrol (Tetrol) specifically induced expression of all three PXR target genes. Because hepatocytes rapidly convert 5β-cholestane-3α, 7α, 12α-triol into bile acids, the intracellular levels of 5β-cholestane-3α, 7α, 12α-triol would be expected to decrease during the course of this experiment. This precludes an accurate analysis of relative efficacy, because 5β-cholestane-3α, 7α, 12α-triol -mediated responses would be underestimated in hepatocyte cultures. Indeed, while 5β-cholestane-3α, 7α, 12α-triol was more efficacious in activating PXR in CV-1 cells as shown in FIG. 3(a), 5β-cholestane-3α, 7α, 12α, 25-tetrol was the more effective activator of hepatocyte specific genes as shown in FIG. 5. Although relative efficacy cannot be determined, these results clearly demonstrate that 5β-cholestane-3α, 7α, 12α-triol and 5β-cholestane-3α, 7α, 12α, 25-tetrol are both effective inducers of endogenous PXR target genes.

### EXAMPLE IV

The liver extract of CYP27 −/− mice shows elevated level of 5β-cholestane-3α, 7α, 12α-triol, enhanced ability to activate mouse PXR. and increased expression of PXR target genes.

Previous studies have reported that β-cholestane-3α, 7α, 12α-triol and 5β-cholestane-3α, 7α, 12α, 25-tetrol levels are elevated in hepatic microsomes of CYP27 null mice and in CYP27 deficient humans with CTX. Honda et al., Side Chain Hydroxylations in Bile Acid Biosynthesis Catalyzed by CYP3A Are Markedly Up-Regulated in Cyp27−/−Mice but Not in Cerebrotendinous Xanthomatosis, J. Biol. Chem. 276: 34579-34585 (2001). It is questioned whether β-cholestane-3α, 7α, 12α-triol (Triol) levels were elevated in whole liver extracts from CYP27 −/− mice. To show the level of 5β-cholestane-3α, 7α, 12α-triol in mouse liver extract, wild-type or CYP27 −/−mouse liver tissue (0.5 g) was extracted with 7.5 ml of ethyl acetate/methanol (2:1, vol/vol) along with 5β-cholestane-3β,5α,6β-triol (25 μg) as an internal control for extraction efficiency. 5β-cholestane-3β,5α,6β-triol does not occur naturally and does not activate mouse PXR (FIG. 3(a)). The extraction was repeated three times, and the organic fraction was pooled and evaporated. To remove the vast excess of cholesterol that interferes with subsequent gas chromatography-mass spectroscopy analysis, the evaporated residue was dissolved in 0.5 ml of chloroform/acetone (35:25, vol/vol) and applied to a Bond Elut SI cartridge (Varian, 500 mg). Cholesterol was removed by washing with 6 ml of chloroform/acetone (35:25, vol/vol), and sterols were eluted with 7 ml of chloroform/acetone/methanol (35:25:20, vol/vol/vol). The sterol fraction was evaporated and dissolved in acetonitrile, and a portion was silylated with N,O-bis(trimethylsilyl)-trifluoroacetamide containing 1% trimethylchlorosilane (Pierce). The silylated material was analyzed by using a Shimadzu model GC-17A gas chromatograph with a QP5000 mass spectral detector and a Hewlett-Packard Ultra 2 (cross-linked-siloxane) column. The injector port was kept at 250° C., interface temperature was kept at 280° C., and oven temperature was kept at 50° C. followed by a gradient of 30° C. min⁻¹ up to 300° C. The electron impact ionization source was at 70 eV. 5β-cholestane-3α, 7α, 12α-triol levels were determined by comparison to a standard curve. For transfection studies, the sterol fraction was dissolved in DMSO and added to the cell-culture media. containing DMEM with 10% delipidated FBS. The dissolved sterol were then added to CV-1 cells transfected with Gal-mouse PXR and reporter genes as described in Example I. The DMSO solution from the wild type and CYP27-null liver was normalized to contain equal amounts of the internal control 5β-cholestane-3β,5α,6β-triol.

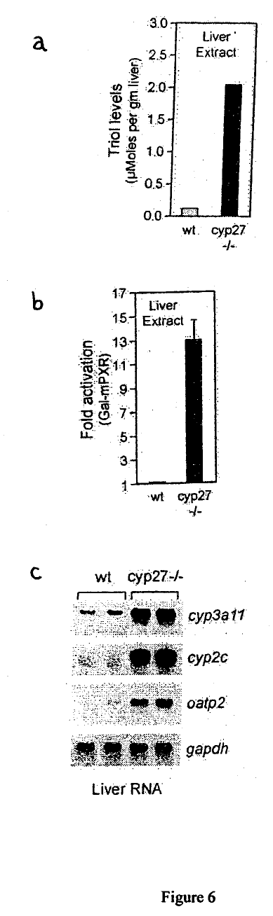

As shown in FIG. 6(a), 5β-cholestane-3α, 7α, 12α-triol (Triol) levels were 16-fold higher in the livers extract of cyp27-null mice than their wild-type counterparts. In the transfection assay where these liver extracts were examined for their ability to activate Gal-mouse PXR in transfected CV-1 cells, the extract from cyp27-null mice induced a 13-fold activation of mouse PXR, whereas extracts from wild-type mice were inactive (See FIG. 6(b)). Furthermore, total RNA was isolated from the livers of wild-type and cyp27-null female mice and Northern analysis was used to measure the expression of PXR target genes according to the methods as described in Example IV. When compared with wild type mice, the expression of cyp3a11, cyp2c, and oatp2 were all dramatically enhanced in the liver of CYP27-null mice (FIG. 6(c)). This effect was specific as the gapdh control transcript was unaffected. Similar results were seen with male mice. These findings demonstrate that the liver extract of CYP27 −/− mice shows elevated level of 5β-cholestane-3α, 7α, 12α-triol which activate mouse PXR in vivo and the expression of PXR target genes.

### EXAMPLE V

Elevated levels of 5β-cholestane-3α, 7α, 12α-triol corresponds to the enhanced drug clearance in CYP27 −/− mice.

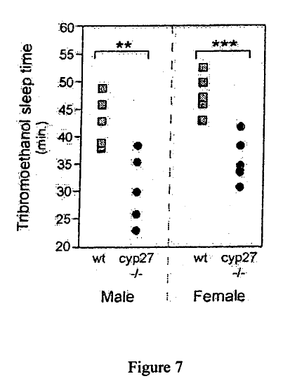

Because PXR is a master regulator of small-molecule clearance, continuous activation of PXR in CYP27-null mice should produce a physiologic state of enhanced resistance to endogenous and exogenous toxins. To test this in a physiological setting, mice received an i.p. injection of the anesthetic agent tribromoethanol (0.35 mg/g of body weight). Drug-induced anesthesia was measured until the animals had regained sufficient consciousness to fully right themselves. Previous studies have demonstrated that sensitivity to tribromoethanol is decreased in mice treated with PXR ligands (Selye, Hormones and Resistance, J. Pharm. Sci. 60:1-28 (1971)) or in transgenic mice with liver-specific expression of a constitutively active PXR chimera (Xie et al., Humanized Xenobiotic Response in Mice Expressing Nuclear Receptor SXR, Nature 406: 435-439 (2000)). Thus, the tribromoethanol-induced sleep test provides a direct and quantitative measure of hepatic PXR activity in live animals. As shown in FIG. 7, male CYP27-null mice were highly resistant to tribromoethanol; they awoke 30.6±2.9 min (mean±SEM) after exposure compared with 42.2±1.9 min for WT controls (P<0.01). In female mice, the difference was even more significant: 35.8±1.6 min for CYP27-null mice compared with 49.6±2.0 min for WT (P<0.001). These results confirm that PXR clearance pathways are highly active in mice with elevated levels of hepatic 5β-cholestane-3α, 7α, 12α-triol. Resistance to tribromoethanol demonstrates that 5β-cholestane-3α, 7α, 12α-triol induced PXR activation effectively protects mice from certain small-molecule toxins. When 5β-cholestane-3α, 7α, 12α-triol itself is accumulating to pathological levels, the ability of this sterol to activate cyp3a11 (FIG. 5) becomes highly significant as CYP3A establishes an alternate or salvage pathway for the elimination of excess 5β-cholestane-3α, 7α, 12α-triol (FIG. 1). Based on the reported Km of 5β-cholestane-3α, 7α, 12α-triol for CYP3A (6 μM; Furster & Wikvall, Identification of CYP3A4 as the Major Enzyme Responsible for 25-Hydroxylation of 5β-cholestane-3α, 7α, 12α-triol in Human Liver Microsomes, Biochim. Biophys. Acta 1437: 46-52 (1999)), this salvage pathway would be initiated as 5β-cholestane-3α, 7α, 12α-triol levels approach the low micromolar range. Since 5β-cholestane-3α, 7α, 12α-triol activates mouse PXR at these same concentrations (FIG. 3(c), EC₅₀≧3 μM), and this in turns leads to enhanced expression of cyp3a11 (FIG. 5), these findings suggest a regulatory loop that minimizes triol accumulation, thereby protecting CYP27 null mice from the pathological consequences of excess sterol accumulation.

### EXAMPLE VI

5β-cholestane-3α, 7α, 12α-triol fails to activate human PXR.

CYP27 deficient or mutant humans develop CTX, a disease characterized by sterol deposits that produce xanthomas, atherosclerosis, gallstones, and neurological dysfunction. The clinical symptoms of CTX in CYP27-deficient humans, but not mice, suggests that humans are unable to shunt into a PXR induced, CYP3A mediated pathway for sterol degradation and elimination. See, FIG. 1. This is an unexpected suggestion in that 5β-cholestane-3α, 7α, 12α-triol is a substrate for both mouse and human CYP3A, and both species of PXR effectively activate the promoters of their corresponding CYP3A genes. Bertilsson et al., Identification of a human nuclear receptor defines a newsignaling pathway for CYP3A induction, Proc. Natl Acad. Sci. USA 95:12208-12213 (1998); Blumberg et al., SXR a novel steroid and xenobioticsensing nuclear receptor, Gene & Dev. 12: 3195-3205 (1998); Staudinger et al., The nuclear receptor PXR is a lithocholic acid sensor that protects against liver toxicity, Proc. Natl. Acad. Sci. USA 98:3369-3374 (2001); Xie et al., Humanized Xenobiotic Response in Mice Expressing Nuclear Receptor SXR, Nature 406: 435-439 (2000).

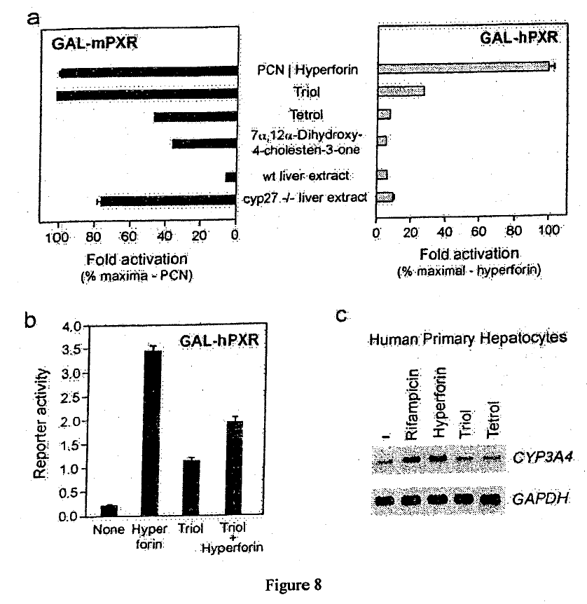

To confirm that humans are unable to take the PXR induced pathway, the ability of the human PXR receptor to respond to 5β-cholestane-3α, 7α, 12α-triol is examined. As shown in FIG. 8(a), in control experiments, 10 μM 5β-cholestane-3α, 7α, 12α-triol (Triol) activated mouse PXR with the same efficiency as optimal amounts of the synthetic agonist PCN. In marked contrast, 5β-cholestane-3α, 7α, 12α-triol displayed very weak activity on the human PXR, which could be fully activated by synthetic ligands such as hyperforin. Similarly, the 5β-cholestane-3α, 7α, 12α-triol precursor, 7α,12α,-dihydroxy-4-cholesten-3-one, and 5β-cholestane-3α, 7α, 12α, 25-tetrol (Tetrol) retained activity on mouse PXR but failed to activate human PXR. These findings demonstrate that specific sterol metabolites that accumulate in CYP27 deficiency fail to activate human PXR. Furthermore, an extract obtained from CYP27-null mouse livers also failed to activate the human PXR. Taken together, these findings demonstrate that human PXR fails to respond to the pool of sterols that accumulate in CYP27 deficiency.

The inability of 5β-cholestane-3α, 7α, 12α-triol to activate human PXR was unexpected because it can interact with the human PXR (FIG. 4). This apparent discrepancy implies that 5β-cholestane-3α, 7α, 12α-triol may function as a partial agonist or weak antagonist of human PXR. Indeed, while hyperforin maximally activates human PXR, the combination of hyperforin and 5β-cholestane-3α, 7α, 12α-triol (Triol) results in suboptimal levels of activation. See, FIG. 8(b). These findings confirm that 5β-cholestane-3α, 7α, 12α-triol is a poor activator or weak antagonist of the human receptor, suggesting that 5β-cholestane-3α, 7α, 12α-triol would not effectively activate CYP3A4-mediated clearance pathways in humans. This is further confirmed in experiments where the expression of PXR target gene CYP3A4 is measured in primary human hepatocytes treated with either 5β-cholestane-3α, 7α, 12α-triol or the synthetic agonists rifampicin and hyperforin. As shown in FIG. 8(c), Indeed, 5β-cholestane-3α, 7α, 12α-triol (Triol) and 5β-cholestane-3α, 7α, 12α, 25-tetrol (Tetrol) had no effect on the expression of CYP3A4, whereas the synthetic agonists rifampicin and hyperforin strongly induced CYP3A4 expression.

Previous studies have identified PXR as a master regulator of xenobiotic clearance. Synold et al., Methods of Modulating Drug Clearance Mechanisms by altering SXR activity. U.S. patent application Ser. No. 09/815,300, filed on Mar. 23, 2001. This designation reflects the receptor's ability to detect a wide variety of foreign compounds and to promote their elimination via a tightly regulated network of xenobiotic metabolizing genes. This regulatory paradigm provides an efficient means to protect the body from potentially toxic foreign compounds. However, it has been unclear whether endogenous PXR ligands exist and what their biological functions might be. It is now demonstrated that excess levels of a naturally occurring cholesterol metabolite (e.g., 5β-cholestane-3α, 7α, 12α-triol) functions as a PXR agonist in mice. These findings extend the role of PXR as an endogenous sterol sensor. Interestingly, 5β-cholestane-3α, 7α, 12α-triol is a key intermediate in the classical pathway of bile acid biosynthesis, which provides the major route for cholesterol degradation in vivo. See, FIG. 1. This pathway converts cholesterol to 5β-cholestane-3α, 7α, 12α-triol which is subsequently metabolized via the enzymatic activity of CYP27 . Thus, individuals that are deficient in CYP27 accumulate high levels of 5β-cholestane-3α, 7α, 12α-trol and ultimately result in the clinical features of CTX. It is now found that 5β-cholestane-3α, 7α, 12α-triol can be metabolized via an alternative PXR induced, CYP3A-mediated pathway in CYP27 −/− mice. The findings in the aforementioned experiments demonstrate that mice respond to excess 5β-cholestane-3α, 7α, 12α-triol by activating mouse PXR and its PXR target gene cyp3a11. This reduces the amount of 5β-cholestane-3α, 7α, 12α-triol that accumulates in CYP27 −/− mice and prevent them from developing CTX-related pathologies.

On the other hand, it is unexpectedly found in the present invention that accumulated 5β-cholestane-3α, 7α, 12α-triol fails to activate human PXR or induce CYP3A4 expression in human hepatocytes. This finding provides a rationale that the failure of 5β-cholestane-3α, 7α, 12α-triol to induce human PXR prevents CYP27 deficient humans from disposing of excess 5β-cholestane-3α, 7α, 12α-triol and therefore lead to the development of clinical manifestations in CTX patients. This finding further provides a rationale to use human agonists to activate human PXR and reduce or eliminate the accumulation of sterol metabolites through a PXR induced, CYP3A activated degradation pathway.

1. A method for enhancing or facilitating the degradation of cholesterol or bile alcohols in a subject in need thereof comprising administering to the subject a pharmaceutically effective dose of a PXR agonist or a PXR agonist composition comprising the PXR agonist and a pharmaceutically acceptable carrier.

2. The method of claim 1 wherein the subject has a condition that can be alleviated by enhancing or facilitating the degradation of cholesterol or bile alcohols and the condition is selected from the group consisting of cerebrotendinous xanthomatosis, cardiovascular diseases, hypertension, atherosclerosis, dyslipidemia, obesity, hypercholesterolemia, hyperlipidemia, hyperlipoproteinemia, hyperchylomicronemia, hyperbetalipoproteinemia, dysbetalipoproteinemia, hyperprebetalipoproteinemia, mixed hyperlipidemia, cholestasis, cholesterolosis, gallstone, cataracts, and hepatomegaly.

3. The method of claim 2 wherein the disorder is cerebrotendinous xanthomatosis atherosclerosis, hypercholesterolemia, hyperlipoproteinemia, dyslipidemia, or hepatomegaly.

4. The method of claim 1 wherein the subject is a human.

5. The method of claim 4 wherein the PXR agonist is a human PXR agonist.

6. The method of claim 4 wherein the PXR agonist composition comprises a human PXR agonist and a pharmaceutically acceptable carrier.

7. The method of claim 5 or 6 wherein the human PXR agonist is selected from the group consisting of dexamethasone t-butylacetate, 11β-(4-dimethylaminophenyl)-17β-phyrdoxy-17α-propinyl-4, 9-estradiene-3-one (RU486, Mifepristone), corticosterone, rifampicin, nifedipine, clotrimazole, bisphosphonate ester SR12813, hyperforin (a component of St. John's wort), paclitaxel (Taxol), ritonavir, lithocholic acid, and 3-keto-lithocholic acid.

8. The method of claim 4 wherein the pharmaceutically effective dose is administered to the human through a administration route selected from the groups consisting of oral, enteral, nasal, topical, rectal, vaginal, aerosol, transmucosal, transdermal, ophthalmic, pulmonary, and parenteral administration.

9. A method of treating a disorder associated with CYP27 deficiency in a subject comprising administering to the subject a pharmaceutically effective dose of a PXR agonist or a PXR agonist composition which comprises the PXR agonist and a pharmaceutically acceptable carrier.

10. The method of claim 9 wherein the disorder associated with CYP27 deficiency is selected from the group consisting of cerebrotendinous xanthomatosis, cataracts, gallstone, tendon xanthomas, atherosclerosis, hepatomegaly, hypertriglyceridemia, and neurological and neuropsychiatric abnormalities such as peripheral neuropathy and dementia.

11. The method of claim 10 wherein the disorder associated with CYP27 deficiency is cerebrotendinous xanthomatosis.

12. The method of claim 9 wherein the subject is human.

13. The method of claim 12 wherein the PXR agonist is a human PXR agonist.

14. The method of claim 12 wherein the PXR agonist composition comprises a human PXR agonist and a pharmaceutically acceptable carrier.

15. The method of claim 12 wherein the PXR agonist is selected from the group consisting of dexamethasone t-butylacetate, 11β-(4-dimethylaminophenyl)-17β-hyrdoxy-17α-propinyl-4, 9-estradiene-3-one (RU486, Mifepristone), corticosterone, rifampicin, nifedipine, clotrimazole, bisphosphonate ester SR12813, hyperforin (a component of St. John's wort), paclitaxel (Taxol), ritonavir, lithocholic acid, and 3-keto-lithocholic acid.

16. The method of claim 12 wherein the pharmaceutically effective dose is administered to the human through a administration route selected from the groups consisting of oral, enteral, nasal, topical, rectal, vaginal, aerosol, transmucosal, transdermal, ophthalmic, pulmonary, and parenteral administration.

17. A method for increasing the degradation of 5β-cholestane-3α, 7α, 12α-triol in a cell from a mammal comprising contacting the cell with a PXR agonist.

18. The method of claim 17 wherein the mammal is a human, a mouse, a rat, or a rabbit.

19. The method of claim 17 wherein the PXR agonist is a human PXR agonist selected from the group consisting of dexamethasone t-butylacetate, 11β-(4-dimethylaminophenyl)-17β-hyrdoxy-7α-propinyl-4, 9-estradiene-3 -one (RU486, Mifepristone), corticosterone, rifampicin, nifedipine, clotrimazole, bisphosphonate ester SR12813, hyperforin (a component of St. John's wort), paclitaxel (Taxol), ritonavir, lithocholic acid, and 3-keto-lithocholic acid.

20. The method of claim 17 wherein the PXR agonist is a mouse PXR agonist selected from the group consisting of 5-alpha-pregnane-3, 20-dione, dexamethasone t-butylacetate, 11β-(4-dimethylaminophenyl)-17β-hyrdoxy-17β-propinyl-4, 9-estradiene-3-one (RU486, Mifepristone), corticosterone, pregnenolone-16α-carbonitrile (PCN), 5β-cholestane-3α, 7α, 12α-triol, 5β-cholestane-3α, 7α, 12α, 25-tetrol, lithocholic acid, 3-keto-lithocholic acid, trans-nonacholar and chlordane, polychlorinated biphenyls, antimineralocorticoid spironolactone, antiandrogen cyproterone acetate, nonylphenol and phthalic acid.

21. A method of treating or preventing a disorder in a subject that can be alleviated by decreasing or inhibiting the degradation of cholesterol or bile alcohols comprising administering to the subject a pharmaceutically effective dose of a PXR antagonist or a PXR antagonist composition which comprises the PXR antagonist and a pharmaceutically acceptable carrier.

22. The method of claim 21 wherein the disorder in a subject that can be alleviated by decreasing or inhibiting the degradation of cholesterol or bile alcohols is selected from the group consisting of hypolipoproteinemia, hypobetalipoproteinemia, and abetalipoproteinemia.

23. The method of claim 21 wherein the PXR antagonist is Esteinascidin-743.

24. The method of claim 21 wherein the pharmaceutically effective dose is administered to the subject through a administration route selected from the groups consisting of oral, enteral, nasal, topical, rectal, vaginal, aerosol, transmucosal, transdermal, ophthalmic, pulmonary, and parenteral administration.
